(19) United States
(12) Patent Application Publication
(10) Pub. No.: US 2025/0152235 A1
(43) Pub. Date: May 15, 2025

(54) SYSTEMS AND METHODS FOR AN IMPLANTED RADIOFREQUENCY ABLATION SYSTEM

(71) Applicant: HELANDE INNOVATIONS, INC., San Diego, CA (US)

(72) Inventors: Kabir Gambhir, San Diego, CA (US); Michael Vaninetti, San Diego, CA (US)

(21) Appl. No.: 18/949,870

(22) Filed: Nov. 15, 2024

Related U.S. Application Data
(60) Provisional application No. 63/599,461, filed on Nov. 15, 2023.
(60) Provisional application No. 63/599,468, filed on Nov. 15, 2023.
(60) Provisional application No. 63/599,476, filed on Nov. 15, 2023.

(51) Int. Cl. A61B 18/14; A61B 17/00; A61B 18/00; A61B 18/12; G16H 10/60; G16H 20/40

(52) U.S. Cl. CPC A61B 18/1477; A61B 18/1206; G16H 10/60; G16H 20/40; A61B 2017/00004; A61B 2017/00199; A61B 2017/00221; A61B 2018/00434; A61B 2018/00577; A61B 2018/00815; A61B 2018/00875; A61B 2018/00898; A61B 2018/1253; A61B 2018/126

(57) ABSTRACT

A wireless radiofrequency ablation (RFA) system for medical treatment, comprising: an implanted system including: a receiving coil configured to wirelessly receive power, and one or more electrodes operably coupled to said receiving coil to deliver thermal energy to a target when powered by the wirelessly received power; and an external control system including: a radiofrequency (RF) generator, a transmission coil configured to wirelessly transmit power to the receiving coil, and a user interface configured to allow transmit power parameters to be input in order to control the RF generator.

### CROSS-REFERENCE TO RELATED APPLICATIONS

This application claims priority to U.S. Provisional Application Nos: 63/599,461, filed Nov. 15, 2023, 63/599,468, filed Nov. 15, 2023, and 63/599,476, filed Nov. 15, 2023.

### TECHNICAL FIELD

The embodiments described herein are generally directed to radiofrequency ablation systems, and more particularly an implantable radiofrequency ablation system.

### BACKGROUND

Radiofrequency ablation (RFA) is a widely used, minimally invasive procedure utilized in various medical applications such as chronic pain management, cardiac arrhythmia treatment, tumor ablation, and endovenous varicose vein treatment, among others. RFA has garnered significant attention in the medical community due to its minimal invasiveness and efficacy in addressing a wide range of health conditions.

The fundamental mechanism of RFA involves applying pulsed or continuous high-frequency electrical currents to modulate, modify, or induce thermal effects in targeted tissue, often with the goal of creating a lesion that alters, inactivates, or destroys tissue at a targeted site. Many procedures involve percutaneously inserting a slender needle electrode into target tissue, typically under the guidance of imaging technologies. In chronic pain management, the standard RFA procedure prevents nerves from transmitting pain signals to the brain. Generally, these types of procedures can be performed on an outpatient basis, taking less than an hour, with patients normally resuming their regular activities within a few days post-procedure.

Chronic pain, a substantial medical issue impacting millions globally and incurring healthcare costs surpassing $560 billion per year in the United States (Dahlhamer et al., 2018), frequently arises from disabling conditions such as spinal facet joint osteoarthritis, a condition characterized by inflammation and pain from the spinal facet joints. These joints are primarily innervated by the lumbar medial branch nerves. Presently, RFA of these nerves, which blocks their ability to signal pain from these joints, stands as a standard treatment for such pain; however, the achieved pain relief is usually temporary, as the ablated nerves typically regenerate over time, restoring pain signaling and necessitating repeated RFA procedures at the same anatomical location.

Traditional RFA systems largely depend on a wired connection between the needle electrode and an external radiofrequency generator. FIG. 1 illustrates the facet joint and the medial branch nerve. FIG. 2 illustrates a conventional wired RFA treatment where needle electrodes are guided by imaging technologies to the joint, where they can then be programmed to ablate the nerve. The necessity of needle electrode placement and adjustment during this wired procedure is time-consuming, painful for the patient, and introduces risks of complications, including infection, bleeding, post-procedure pain, and unintentional injury to adjacent tissue or nerves. Each subsequent ablation procedure re-exposes patients to these risks.

### SUMMARY

Systems and methods for radiofrequency ablation systems are described herein.

According to one aspect, a wireless radiofrequency ablation (RFA) system for medical treatment, comprises: an implanted system including: a receiving coil configured to wirelessly receive power, and one or more electrodes operably coupled to said receiving coil to deliver thermal energy to a target when powered by the wirelessly received power; and an external control system including: a radiofrequency (RF) generator, a transmission coil configured to wirelessly transmit power to the receiving coil, and a user interface configured to allow transmit power parameters to be input in order to control the RF generator.

### BRIEF DESCRIPTION OF THE DRAWINGS

The details of embodiments of the present disclosure, both as to their structure and operation, may be gleaned in part by study of the accompanying drawings, in which like reference numerals refer to like parts, and in which:

FIG. 1 illustrates the facet joint and the medial branch nerve;

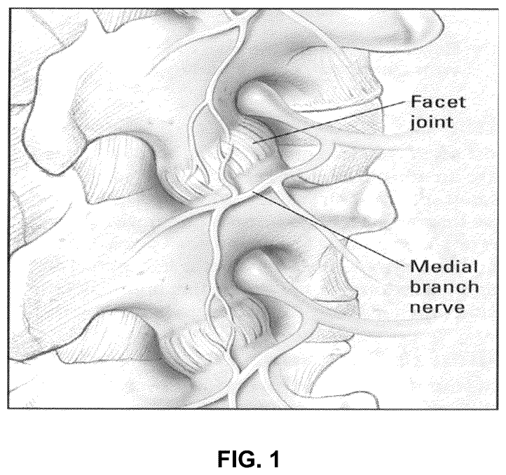

FIG. 2 illustrates a conventional wired RFA treatment;

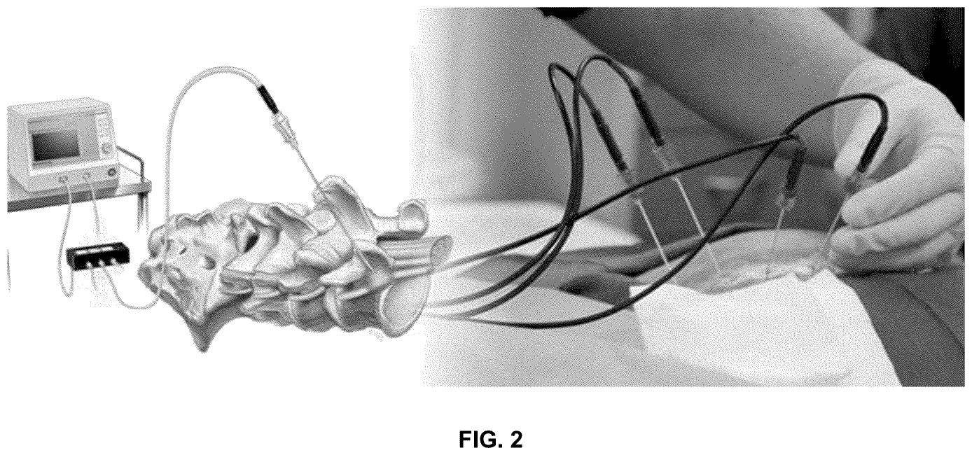

FIG. 3 illustrates a simplified block diagram of a treatment system, according to an embodiment;

FIG. 4 illustrates the control feedback loop that can be implemented in the system of FIG. 3, according to an embodiment;

FIG. 5 is a diagram that illustrates the treatment system of FIG. 3 in more detail, according to one embodiment;

FIG. 6 illustrates that for spinal facet joint osteoarthritis, the leads of the system of FIG. 3 can be positioned on or near the medial branch nerve;

FIG. 7 illustrates an example infrastructure, in which one or more of the processes described herein, may be implemented, according to an embodiment; and

FIG. 8 illustrates an example processing system, by which one or more of the processes described herein, may be executed, according to an embodiment.

### DETAILED DESCRIPTION

The detailed description set forth below, in connection with the accompanying drawings, is intended as a description of various embodiments, and is not intended to represent the only embodiments in which the disclosure may be practiced. The detailed description includes specific details for the purpose of providing a thorough understanding of the embodiments; however, it will be apparent to those skilled in the art that other embodiments can be practiced without these specific details. In some instances, well-known structures and components are shown in simplified form for brevity of description.

It should also be understood that the various components illustrated herein are not necessarily drawn to scale. In other words, the features disclosed in various embodiments may be implemented using different relative dimensions within and between components than those illustrated in the drawings.

The systems and methods for wireless RFA disclosed herein eliminate the need to insert a needle electrode for each treatment. Instead, an implanted electrode coupled with a receiving coil as described below, is placed one time at the target tissue site, and RF energy is wirelessly coupled to the electrode to ablate the nerve tissue or other target transcutaneously. Once the electrode(s) and receiving coil are implanted, subsequent procedures/treatments can be significantly faster than conventional procedures/treatments that use a wired approach, and can be repeated as often as needed with the original implanted electrode.

Moreover, certain embodiments described herein use impedance sensing and control algorithms to augment the efficiency and effectiveness of RFA procedures, thus facilitating personalized, longer-lasting treatment for, e.g., chronic back pain, and ultimately, enhanced patient outcomes. This presents a considerable improvement over conventional RFA and wireless implant techniques by enabling procedure optimization based on tissue feedback, thereby ensuring safe and therapeutic relief from chronic pain. Through the correlation of impedance to temperature and ablation effects, as described below, the system can intelligently dictate the necessary power levels and timing to achieve complete, e.g., denervation of the targeted nerve or tissue ablation. This enables repeat RFA treatments to be conducted wirelessly and non-invasively, using a wireless power transfer mechanism combined with impedance monitoring. This enhances patient comfort while increasing the accuracy and precision of subsequent RFA procedures.

As noted, the systems and methods described herein provide the ability to monitor and dynamically control the ablation process through real-time impedance sensing. As the tissue temperature increases during energy delivery, its impedance decreases due to increased ion mobility. This causes the total load impedance of the receiving coil circuit to decrease proportionally. By monitoring the receiving coil impedance in real-time, the changes can be correlated with temperature increases at the electrode-tissue interface.

Feedback control algorithms within the external control system determine the appropriate frequency and levels of RF energy for delivery through the electrodes based on the impedance and temperature data. A user interface (U/I) connected to the external control system allows the physician or other professional to select parameters like conventional or pulsed ablation, frequency, power level, and duration. The interface displays impedance, temperature, power, and system status data during the procedure. Manual adjustments are possible within integrated safety ranges.

In an embodiment for chronic back pain treatment, the system provides wireless power to an implanted receiving coil and bipolar electrode array placed along the medial branch nerves. Automated impedance feedback enables real-time control of the power and frequency levels, ensuring adequate energy is delivered for safe and complete medial branch ablation.

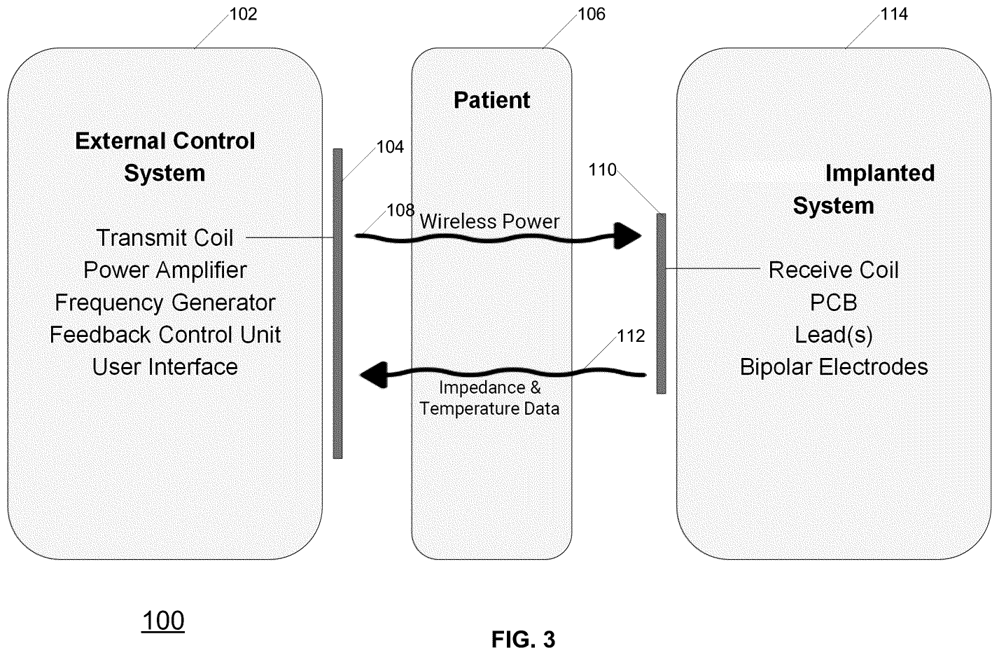

FIG. 3 is a simplified block diagram of a treatment system 100 configured in accordance with one example embodiment. As can be seen, system 100 comprises an external control system 102 that interfaces with a transmission coil 104 outside of patient 106. The external control system can comprise a frequency generator and power amplifier to generate wireless power 108 that is inductively coupled with a receiving coil 110 on the inside of patient 106. External control system 102 can also comprise a feedback control unit to receive impedance/temperature data 112 from the implanted system 114 inside of the patient and interfaced with the receiving coil 110. The implanted system 114 can comprise the leads and what in this example embodiment would be bipolar electrodes that are positioned on or near, e.g., the medial branch nerve or other target. It should be noted that monopolar or multipolar electrodes can also be used with the embodiments described herein. Note that a grounding mechanism may need to be included for monopolar implementations. For example, a return electrode configured to be positioned on the patient's skin to complete the electrical circuit during ablation.

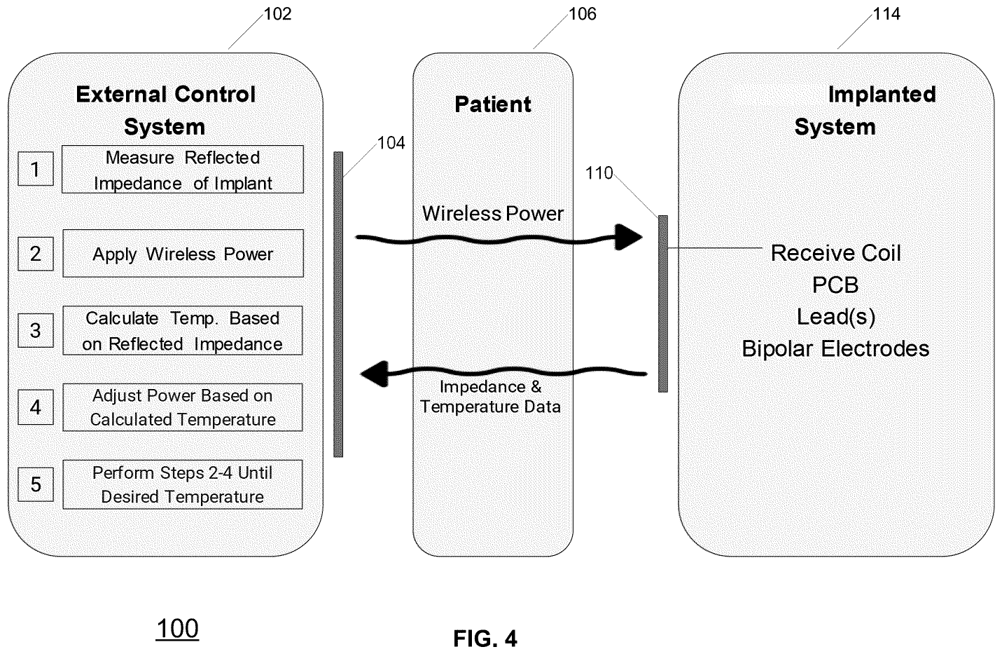

FIG. 4 illustrates the control feedback loop that can be implemented by system 100 as described above. As can be seen, in step (1) the reflected impedance of the receiving coil 110 can be measured before the treatment begins. Then in step (2), wireless power can be applied to the receiving coil 110 via transmission coil 104. External control system 102 can then calculate the temperature of the target site in step (3) based on the reflected impedance. In step (4) the power can be adjusted if needed based on the calculated temperature. In step (5), the process can be repeated to maintain the desired temperature. For example, the external control system 102 can comprise a microprocessor and memory, as described below, containing feedback control algorithms that control the power based on the calculated temperature.

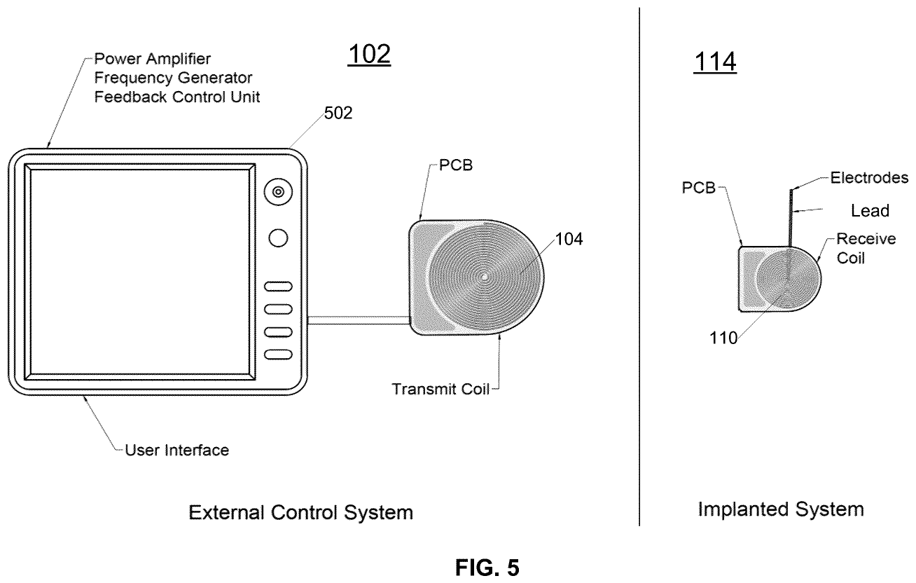

FIG. 5 is a diagram that illustrates the treatment system 100 in detail. First, as can be seen, the external control system 102 can comprise a control unit 502 that includes a User Interface (U/I) that includes a display and input and output controls such as buttons, toggles, speakers, microphone, etc. Control unit 502 can be coupled with transmission coil 104 as illustrated and can include the frequency generator, power amplifier and feedback control. Alternatively, some or all of these components of system 102 can be external from the U/I.

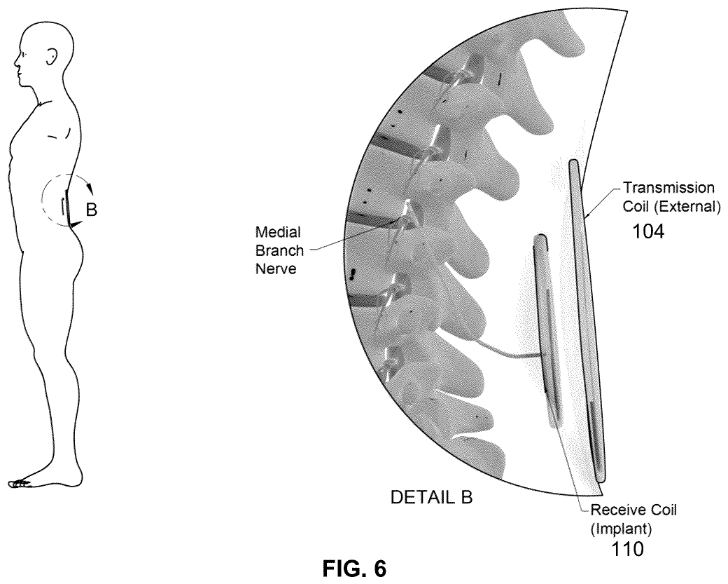

The transmission coil 104 can then be inductively coupled with the receiving coil 110 as described above. Receiving coil 110 can be coupled with the leads that position the electrodes at the target site. FIG. 6 illustrates that for spinal facet joint osteoarthritis the leads can position the electrodes on or near the medial branch nerve.

Thus, the receiving coil 110 can be implanted with the electrodes appropriately positioned. This requires one invasive procedure. Subsequently, the patient can come in for treatment, have the transmission coil 104 positioned outside the body to inductively couple with the receiving coil 110 and deliver the energy needed to ablate the medial branch nerve(s) using the feedback control loop of FIG. 4.

It should be noted that the receiving coil 110 can be configured to drive multiple leads to treat multiple sites. In order to accomplish this, a switching mechanism is included, such as reed switches, magnetic switches, hall effect sensors, etc., to control which leads are activated.

Thus, by way of example only and not limitation, the implanted system 114 can comprise: A receiving coil 110 implanted under the skin to receive wireless power at, e.g., 1 Hz to 1 MHz. It will be understood that other frequencies and frequency ranges can also be used, such as 6.78 MHz and 13.56 MHz, which are Industrial, Scientific, and Medical (ISM) standard frequencies. The receiving coil 110 can be a planar spiral coil, from 5 mm to 50 mm in diameter, e.g., about 25 mm in diameter, wound with about 15 turns of 20 AWG polyimide insulated copper wire.

An array of multiple electrodes, preferably two electrodes, can be spaced 1-10 mm apart, e.g., about 3 mm apart, and made from a conductive alloy, such as platinum-iridium. A pair of bipolar annular electrodes, 0.5 mm to 10 mm in diameter, e.g., about 3 mm in diameter, 2 mm to 10 mm in length, and, e.g., about 5 mm long, that are coaxially located along the distal end of an insulated lead assembly. In other embodiments, the lead length is between about 1-30 cm. The leads can for example have a length of 5-30 cm, e.g., about 15 cm, and can be made from conductive alloy wires, such as nitinol, 316 stainless steel, MP35N, or platinum-iridium. The leads can be coated in a flexible polymer to ensure biocompatibility.

By way of example only and not limitation, the external control system 102 can comprise a transmission coil 104, 25 mm to 500 mm diameter, e.g., about 100 mm diameter, about 20 turns of 14 AWG polyimide insulated copper wire, with the ability to transmit RF energy and signals. Control system 102 can also comprise an RF signal generator and power amplifier to generate an RF signal at a fixed frequency, e.g. 500 kHz for continuous and 500 kHz for pulsed with an active cycle of 20 ms and an inactive cycle of 480 ms, with an adjustable power output level from 0-10 W. While continuous temperatures typically don't exceed about 80° C., for pulse operation the limit is about 42° C. The RF signal and power generator can have the ability to measure the impedance of the implanted system 114 prior to implantation and measure impedance during the nerve ablation procedure.

A feedback control unit implemented via a microprocessor and memory system as described below can be configured to run algorithms to determine appropriate RF power levels based on real-time impedance data which can be correlated to temperature data.

An, e.g., touchscreen user interface can display system status and alerts and provide the ability to select, e.g., conventional or pulsed ablation, frequency, input power, waveform, and duration of treatment. Other displayed information can include impedance, temperature, implant receive power, efficiency, and transmission output power. Manual control and adjustment options can also be available through the user interface, but feedback algorithms can as noted automatically modulate power for safety, optimal results, and therapeutic endpoints.

The procedure setpoints can be changed manually via the U/I by a physician within a range of safety limits integrated into the system. Depending on the embodiment, some or all of the parameters and setpoints can be adjusted as needed, including but not limited to rate of heating, power, frequency, temperature, and projected lesion size. As such, a physician can perform adjustments to ensure the best therapeutic outcomes, based on a patient's physiology and diagnosis.

An example of an external control system 102 providing power to an implanted system 114 follows for a continuous (conventional) ablation. The clinician can set the initial parameters to control the tasks of applying power, measuring temperature, maintaining power at temperature set point, and reducing power assuming the implanted system 114 is in place and the electrodes are positioned next to the tissue requiring ablation.
1. Position and align transmission coil over implanted receiving coil, where receiving coil is located about 5-15 cm from target nerve and leads extend to electrodes positioned next to nerve.
2. Perform sensory nerve and motor nerve testing to verify electrode placement is not adjacent to tissue or nerves not requiring ablation.
3. Set parameters of external control system: For example, 90 seconds, at about 80° C. target temperature (e.g. reflected impedance of ≤9Ω correlates with 80° C.).
4. Initialize system. Measure initial reflected impedance (assume 12Ω). Set initial power to 1 W.
5. Set initial power increase amount (e.g. 0.5 W) and time interval (e.g. 5 sec).
6. Enter main control loop:
   a. Measure reflected impedance
   b. If reflected impedance>11Ω, increase power by set amount every interval
   c. If 9Ω<reflected impedance≤11Ω, reduce amount and interval (e.g. 0.25 W, 2 sec)
   d. If reflected impedance≤9Ω, hold power constant
7. Maintain constant power for 90 seconds once reflected impedance reaches≤9Ω
8. Reduce power and monitor impedance rise to ensure cooling before removing transmission coil.

The following describes example treatments that can be performed using system 100.

### Chronic Back Pain Treatment

In some embodiments, as described above, the implanted system 114 comprises the receiving coil and leads, with the power source, control circuitry, while the monitoring and control is performed by external control system 102, which is entirely external to the patient. The reduction in implanted components enhances safety and reliability while still enabling automated impedance-based control of treatment parameters through reflected power sensing via implanted system 114 as described above.

In some embodiments, the external control system 102 can be programmed to estimate the post-ablation impedance curve based on calibration studies in specific tissue types. By forecasting expected impedance changes as the ablated tissue heals and providing this information to physicians, the system 100 can improve treatment planning for repeat ablation procedures where needed.

In some embodiments, an array of thermistors (not shown) can be positioned near the electrodes and connected to the receiving coil 110 circuitry. As the thermistors experience increasing temperatures during the ablation procedure, their resistance decreases proportionally, creating discrete changes in the overall load impedance of the receiving coil 110 circuit. The external control system 102 detects these impedance changes in the reflected signals from the receiving coil 110, which can then be correlated to parameters like tissue temperature.

In some embodiments, optional temperature monitoring equipment, e.g., included in external control system 102, can measure temperature at the electrodes. The temperature monitoring equipment can be powered by an internal receiving coil, which is also powered wirelessly from the transmission coil and external control system. This coil can for example be receiving coil 110. But in certain embodiments, a secondary implanted coil or antenna (not shown) can be included, which is connected to a thermistor by a secondary set of electrodes located adjacent to the primary electrodes attached to the leads. The thermistor can then measure redundant temperature information which would increase the precision and safety of the primary ablation system.

As noted, in some embodiments, such a secondary temperature monitoring thermistor can be incorporated into the primary leads connecting the internal receiving coil 110 to the treatment electrodes. One or more thermistors can be incorporated adjacent to the electrodes to sense tissue temperature at the target site.

In some embodiments, monitoring equipment can measure temperature at the electrodes and tissue at the target site by utilizing a temperature monitor and communication device. Wireless connectivity provides more deployment and use options as well as tapping into the potential to enhance procedures through connected devices. The temperature monitor and communication device (not shown), also known as a wireless body area network (WBAN), can be integrated or placed separately in relation to the implanted system 114. The temperature monitoring equipment can utilize various low power communication protocols, including Bluetooth, Bluetooth Low Energy, Near Field Communication (NFC), Radio Frequency Identification (RFID), Ultra-Wide Band (UWB), Near Field Magnetic Induction (NFMI), and Rubee. For example, a battery powered Rubee sensor can be configured with single or multiple thermistors attached near to the electrodes to monitor and wirelessly communicate temperature data to an external device, e.g., the external control system 102, during a procedure. The Rubee sensor could also measure temperature over a period of time and record this information in memory for future access.

In some embodiments, system 100 includes an access port and catheter lumen which are integrated into the implanted system 114, enabling infusion of analgesics, lidocaine, or other therapeutic liquids to the desired site, such as at or near the catheter tip. During implantation, lidocaine infusion through the port can provide temporary numbness to confirm proper positioning and pain relief during the RFA procedure. Subsequently, immediately prior to repeat RF procedures, particularly high temperature RF, analgesic can be administered via this lumen to reduce or prevent pain during RF treatment. A templated guide can facilitate the alignment on the skin with the housing to indicate the port's location for percutaneous access. This enables clinicians to inject fluids without visually confirming the port site. Analgesics can also be delivered post-operatively after implantation, post repeat ablation, or even between RF treatments through the port for supplementary pain management. The wireless power transfer and impedance monitoring capabilities remain fully functional during infusion through this embodiment's port and lumen. This provides the added utility of drug delivery while maintaining the core functionality of the system.

### Cancer Tumor Ablation

In some embodiments, system 100 can be used to thermally ablate tumors through wireless energy delivery to bipolar or multipolar electrodes placed at targeted locations under image guidance. Measuring changes in impedance during heating, as described above, provides feedback on tissue destruction for automatically controlling ablation such that an entire tumor volume receives adequate thermal dose. The non-invasive powered approach helps avoid complications from wired connections traversing healthy tissues surrounding the tumor. Repeat wireless ablation of recurrent tumors is also possible based on the system's configuration and capabilities.

### Hypertension Treatment

In some embodiments, system 100 can be adapted to perform renal denervation through precision ablation of the renal sympathetic nerves. By accessing the renal arteries percutaneously and placing wireless powered electrodes under image guidance at locations where renal nerves are closely associated, focused and highly controlled RF energy can be delivered based on automated feedback from impedance sensing. Varying power levels and heating durations based on nerve density and position helps maximize ablation of sympathetic renal nerves for antihypertensive effect while limiting damage to adjacent tissue. The minimally invasive and non-invasive nature of the wireless approach may reduce risks of renal artery injury or limited efficacy compared to existing renal denervation techniques.

### Chronic Pancreatitis Pain Relief

In some embodiments, system 100 can be used to ablate the celiac plexus which can provide palliation caused by intractable intraabdominal pain due to malignancy or chronic pancreatitis. Ablating these nerves via injecting alcohol or wired ablation can provide pain relief for three to six months. System 100 can provide repeated wireless ablations of the celiac plexus, providing routine outpatient palliative care. The wireless non-contacting nature of the system can provide a more rapid and repeatable approach to pain relief. This also can reduce a patient's dependency on opioids or non-opioid analgesics.

### Wireless Nerve Ablation

In some embodiments, the wireless power transfer and dynamic control functionality described above can be directed at nerve targets beyond the medial branches, such as the vagus nerve or splanchnic ganglia for certain disorders. Precision nerve ablation through implanted system 114 with feedback control, as described above, allows for targeted denervation of specific nerves implicated in conditions like gastroparesis, pancreatitis, hypertension or depression. The automated and adjustable nature accommodates differences in individual anatomy to optimally ablate the relevant nerves for maximum benefit. Repeat procedures are possible to achieve and maintain the desired neurolytic effects.

### Dorsal Root Ganglion Treatment

In some embodiments, system 100 can be surgically positioned along the dorsal root ganglia near the spinal column to enable wireless pulsed radiofrequency (PRF), thermal ablation, or stimulation treatment. The system can provide PRF to the dorsal root ganglia, modulating pain signaling without fully ablating the tissue.

### Peripheral Nerve Treatment:

In some embodiments, system 100 can deliver PRF, high-temperature RF, electrical stimulation, or modulation directed towards peripheral nerves to treat acute or chronic pain. System 100 can also provide muscle conditioning or reconditioning via muscle activation via stimulation of motor nerve fibers.

### Nerve Conduction Block

In some embodiments, system 100 can be leveraged for reversible nerve conduction block through stimulation rather than ablative modalities. Blocking nerve signals for diagnostic or therapeutic reasons without permanent destruction of nerves can be achieved by employing the feedback capabilities to determine power levels and pulse parameters necessary to temporarily block target nerve potentials. The effects of specific nerve blockades can be evaluated without committing to permanent denervation in cases where nerve function may potentially recover or be desirable to restore in the future.

### Neural Pathway Activation

In some embodiments, the wireless capabilities of system 100 can be directed at weaker or non-thermal stimulation of nerves for therapeutic benefit. Power levels and pulse stimulations are precisely tuned to activate certain neural pathways or modulate signaling for targeted effects. Feedback from real-time impedance monitoring helps determine ideal stimulation parameters to achieve long-term and repeatable activation of desired neural circuits without spread of effects to neighboring regions. Examples include stimulation of the phrenic nerve for central sleep apnea or sacral nerve stimulation for bowel/bladder control.

### Spinal Cord Stimulation

In some embodiments, the non-invasive delivery of wireless energy via system 100 can power temporary stimulation leads placed in the epidural space of the spine. The wireless powered and automatically adjustable stimulations from these leads aim to override pain signaling for diagnostic purposes in determining if a patient may benefit from spinal cord stimulation for certain pain conditions before committing to a permanent system. Impedance sensing provides feedback control of power levels, allowing the simulation of various stimulation locations, intensities and programming options to evaluate outcomes.

In some embodiments, the wireless power transfer of system 100 can be adapted to enable paresthesia-free spinal cord stimulation by positioning leads in the dorsal horn where high-frequency stimulation may block pain signals without inducing numbness or tingling sensations. The automated control capabilities are applied to precisely regulate power levels, pulse widths and frequencies delivered to the dorsal horn region for maximum pain relief utilizing stimulation at 300-1000 Hz with certain pulse characteristics may provide therapeutic benefit without paresthesia. Impedance sensing allows feedback-guided positioning to target the proposed mechanism of action in the dorsal horn. This approach can expand and customize spinal cord stimulation (SCS) options for patients seeking to eliminate pain without side effects like paresthesia.

### Wireless Lead Detachment

In some embodiments, the wireless power transfer and control offered by system 100 can be used to safely and automatically disconnect medical implants following use. For example, leads used for trial spinal cord stimulation that are temporarily placed in the epidural space for assessment of potential benefits can be detached wirelessly on request by modulating the power waveform to dissolve biodegradable adhesives or junctions selectively. This helps avoid the risks of manual lead or device explant procedures to remove the trial system. By designing the leads and connections between components to be sensitive to certain power frequencies or energy levels, the intact system can remain securely in place until intentionally disconnected as needed using the wireless link.

### Wireless Drug Delivery

In some embodiments, the capabilities of system 100 can be integrated into implantable infusion pumps to enable automated, feedback-controlled targeted drug delivery without percutaneous tubing. Wireless energy can power the pump to expel drugs from a reservoir into miniature microfluidic channels leading to infusion outlets at specific anatomical targets. Sensing changes in the flow or backpressure within the channels using reflected power provides feedback to adjust pump rate/flow and achieve the desired drug dosage in a precise area. Potential applications include chemotherapeutic agents for malignant tumors, autoantibodies for certain conditions, or simply acting as a wireless extension of existing pump technology to enhance mobility and outcomes.

### Implanted Sensor Connectivity

In some embodiments, the wireless power capabilities of system 100 can provide energy to implanted sensors, monitoring vital signs or physiological parameters to enable automated, rapid adjustments of treatments through a connected feedback loop. For example, blood chemistry sensors can be used to determine if levels of a particular compound have changed, signaling an implanted insulin or drug pump to wirelessly adjust its output rate in real time as needed to maintain levels within a target range. Vital sign metrics like heart rate, blood pressure or respiration can also be monitored by body-worn or implanted sensors with data and power provided wirelessly to enable continuous feedback and tight control loops for optimally tailored therapies. Wireless connectivity helps overcome issues like broken sensor leads or inability to access percutaneously connected components and enhances patient mobility during monitoring.

### Vagus Nerve Modulation

In some embodiments, the wireless capabilities of system 100 can be directed at neuromodulation of the vagus nerve for patients with autoimmune inflammatory disorders. Precision neural stimulation is used to activate vagus nerve pathways involved in regulating and balancing the body's immune and inflammatory responses. Power levels and pulse parameters are automatically adjusted based on electrode impedance feedback to identify amplitudes for targeted stimulation of relevant vagal fibers without spread of effects to neighboring regions. Neuromodulation aims to beneficially alter immune function for conditions like rheumatoid arthritis, inflammatory bowel disease or lupus without the side effects of broad anti-inflammatory drugs. Repeat use or touch-up procedures are possible as needed to achieve and maintain therapeutic impacts.

### Chronic Migraine Pain Relief

In some embodiments, system 100 can be adapted for targeted ablation of peripheral nerves implicated in chronic migraines, such as the greater occipital nerve. Wireless powered electrodes are percutaneously implanted under the guidance of peripheral nerve imaging for accurate positioning in proximity to the target nerve. Impedance sensing and feedback control capabilities are then used to precisely deliver RF energy and ablate the nerve to alleviate migraine pain based on established dosing guidelines while accounting for individual patient anatomy. A minimally invasive, non-invasive approach with dynamic control of ablation may help improve positive response rates compared to percutaneous RFA procedures for migraine. Repeat use for recurrent neuropathic headaches is enabled by the wireless capabilities.

### Cluster Headache Pain Relief

In some embodiments, system 100 can be adapted to modulate signaling in the sphenopalatine ganglion (SPG) through localized stimulation or radiofrequency ablation for management of severe chronic cluster headache pain. The SPG has been shown to play a role in transmitting aberrant signals involved in cluster headache attacks. Placement of wireless-powered electrodes in the area of the SPG using minimally invasive techniques allows for either temporary blocking of SPG signaling via stimulation or longer disruption through precision radiofrequency ablation based on real-time impedance feedback. Automating the neurolysis or neuromodulation procedure enables consistent and individually tailored treatment at the difficult to access SPG region. Repeat procedures when the effects of ablation or stimulation wane over months may extend benefits.

### Splanchnic Nerve Modulation

In some embodiments, the wireless capabilities of system 100 can be leveraged for neuromodulation of the splanchnic nerves to manage upper gastrointestinal (GI) symptoms like nausea, bloating or pain commonly associated with gastroparesis or other motility disorders. Precision wireless stimulation of these nerves modulates signaling patterns to alleviate problematic GI effects and slow gastric emptying to a more normal and tolerable rate based on sensor feedback. By normalizing nerve function and gut-brain communication in this manner, quality of life can be improved for patients with upper GI neuromuscular conditions.

### Sleep Apnea Treatment

In some embodiments, the wireless stimulation capabilities of system 100 can be applied to the phrenic nerve for treatment of central sleep apnea by activating the diaphragm during periods of apnea to restore normal breathing. A frequency range of 10-40 Hz is utilized for effective phrenic nerve engagement. Impedance sensing provides feedback on lead positioning and stimulation thresholds to identify parameters for selectively activating the phrenic nerve. Automated adjustment of frequency, amplitude, and pulse width helps ensure stimulation spreads to the diaphragm without excessive activation of surrounding tissue. The wireless stimulation approach eliminates issues with wire breakage or disconnection, enhancing reliability for long term use.

### Temporary Peripheral Nerve Stimulation

In some embodiments, system 100 can be adapted to provide temporary peripheral nerve stimulation for the assessment of potential benefits prior to permanent implantation. Wireless powered leads are placed in proximity to target peripheral nerves, such as the occipital, vagus or pudendal nerves, using minimally invasive techniques. The stimulation parameters are then automatically adjusted based on impedance feedback to provide nerve activation for a trial period of days or weeks while evaluating the patient's response. If adequate and sustained pain relief or other therapeutic impacts are observed, the temporary leads can be removed and replaced with a long-term stimulator if desired.

### Tremor or Movement Disorder Treatment

In some embodiments, the wireless capabilities of system 100 can be used independently or in addition to standard treatments for tremor or movement disorders such as essential tremor or Parkinson's disease. Targeted modulation of signaling in certain neural pathways involved in motor control is achieved through precision wireless stimulation based on real-time impedance feedback to optimize power levels for desired effects. For example, stimulation of the ventral intermediate nucleus may help reduce tremor severity. A wireless method for activating specific neural targets at multiple sites in certain movement disorders avoids issues with wire breakage or poor connectivity and may enhance mobility/function when used for tremor management whether alone or complementing drugs and surgery. Repeat long term use is possible as needed to continue providing benefits.

### Chronic Pain Treatment

In some embodiments, system 100 can be utilized to wirelessly stimulate or lesion peripheral nerves in precise patterns to achieve desired neural reprogramming for chronic pain. By modifying signaling between the peripheral and central nervous system in a methodical stepped fashion through a series of procedures over weeks or months, maladaptive pain pathways can potentially be redirected toward normal processing and modulation. The automated and wireless capabilities allow for closely controlled delivery of stimulation/ablation energy to peripheral nerves based on impedance feedback at specific sites in accordance with reprogramming protocols that aim to reverse pain as a disease state. The gradual effects from neural reorganization may provide longer-term relief than temporary stimulation alone by treating underlying pain mechanisms.

### Acute Pain Management

In some embodiments, system 100 can be used to stimulate peripheral sensory nerves to mask or block pain signals for acute pain management in a hospital or clinical setting. Temporary wireless leads are placed in proximity to target sensory nerves, such as the median, ulnar or posterior tibial nerves under ultrasound guidance. Automated impedance feedback is then used to determine appropriate stimulation levels to activate sensory nerve fibers and induce paresthesia in the affected region, masking the patient's perception of pain for a period of hours.

### Pelvic Floor Dysfunction Treatment

In some embodiments, the wireless stimulation capabilities of system 100 can be directed at the pudendal nerve to restore certain pelvic floor functions impaired by childbirth or aging. Precision stimulation utilizing impedance feedback automatically determines power levels necessary to activate relevant pudendal nerve pathways involved in urethral sphincter control and fecal continence without inducing paresthesia. By selectively stimulating the appropriate neural circuits of the pudendal nerve in either an acute or chronic manner, bladder and bowel control can potentially be re-established as a non-invasive therapeutic option. Wireless temporary or long-term use avoids issues with poor connectivity/broken leads in perineal regions. Repeat use may be needed to maintain benefits, depending on the underlying condition.

### Complex Regional Pain Syndrome Treatment

In some embodiments, system 100 can be adapted to ablate hyperactive sympathetic nerves associated with complex regional pain syndrome (CRPS). Under imaging guidance, wireless-powered bipolar electrodes are implanted in proximity to the lumbar sympathetic chain and lumbar splanchnic nerves which carry signals linked to pain, inflammation and vasomotor dysfunction in complex regional pain syndrome (CRPS). Precision radiofrequency ablation is then carried out via the wireless electrodes based on real-time impedance feedback to identify and disrupt these hyperactive sympathetic pathways in a controlled manner while avoiding damage to neighboring tissue. Selective denervation of specific sympathetic fibers aims to reduce severe pain associated with CRPS and potentially restore some normal function to the affected limb. The minimally invasive and adjustable wireless approach can limit morbidity compared to surgical sympathectomy. Repeat use can re-ablate regenerating sympathetic fibers as needed to provide ongoing relief.

In certain embodiments, transmission coil 104 can be connected via some form of wire or cable with an external control system 102 that is positioned near transmission coil 104. In other embodiments, transmission coil 104 can be wireless connected with external control system 102. This would require that the PCB associated with the transmission coil 104 (see FIG. 5) comprise the wireless communication circuitry necessary for such. This would also require the drive circuitry, e.g., the frequency generator and power amplifier, for transmission coil 104 to be on the associated PCB, or at least the drive circuitry would need to be connected to transmission coil 104.

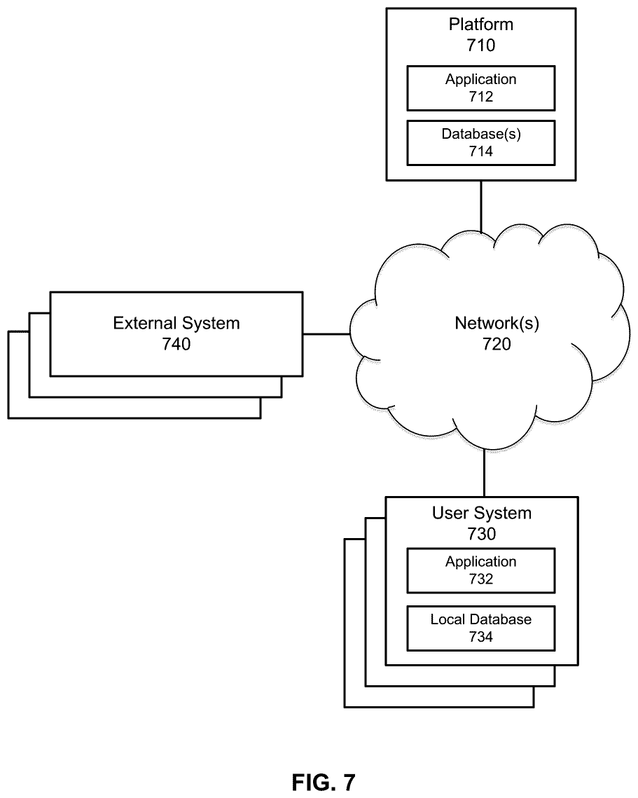

The ability to interface wirelessly with the transmission coil 104 can allow the external control system 102 to be remote from the transmission coil 104, which in turn can allow embodiments where multiple transmission coils can be controlled remotely using a single external control unit. In fact, the control system can be completely remote and implemented as a platform 710 as described below with respect to FIG. 7. In any such remote embodiment, some form of external control system may still reside locally, and/or be interfaced with transmission coil 104, and act as a user system 730.

In certain embodiments multiple implanted systems 114 can be implanted in a patient. In such an embodiment, rather than a single implanted system 114, possible with switches that would enable multiple electrodes at different location as described above, multiple implanted systems 114 can be implanted to treat various areas requiring ablation, e.g. three implants could be spaced a few inches apart and the leads extending from each implant would be placed at medial branch nerve areas located at three unique places along the spine.

In certain other embodiments, an ultrasound transducer can be used as the power source, i.e., in place of transmission coil 104, which the implanted system 114 can then convert to RF energy to drive the electrodes.

FIG. 7 illustrates an example infrastructure in which one or more of the disclosed processes may be implemented, according to an embodiment. The infrastructure may comprise a platform 710 (e.g., one or more servers) which hosts and/or executes one or more of the various processes, methods, functions, and/or software modules described herein. Platform 710 may comprise dedicated servers, or may instead be implemented in a computing cloud, in which the resources of one or more servers are dynamically and elastically allocated to multiple tenants based on demand. In either case, the servers may be collocated and/or geographically distributed. Platform 710 may also comprise or be communicatively connected to a server application 712 and/or one or more databases 714. In addition, platform 710 may be communicatively connected to one or more user systems 730 via one or more networks 720. Platform 710 may also be communicatively connected to one or more external systems 740 (e.g., other platforms, websites, etc.) via one or more networks 720.

Network(s) 720 may comprise the Internet, and platform 710 may communicate with user system(s) 730 through the Internet using standard transmission protocols, such as HyperText Transfer Protocol (HTTP), HTTP Secure (HTTPS), File Transfer Protocol (FTP), FTP Secure (FTPS), Secure Shell FTP (SFTP), and the like, as well as proprietary protocols. While platform 710 is illustrated as being connected to various systems through a single set of network(s) 720, it should be understood that platform 710 may be connected to the various systems via different sets of one or more networks. For example, platform 710 may be connected to a subset of user systems 730 and/or external systems 740 via the Internet, but may be connected to one or more other user systems 730 and/or external systems 740 via an intranet. Furthermore, while only a few user systems 730 and external systems 740, one server application 712, and one set of database(s) 714 are illustrated, it should be understood that the infrastructure may comprise any number of user systems, external systems, server applications, and databases.

User system(s) 730 may comprise any type or types of computing devices capable of wired and/or wireless communication, including without limitation, desktop computers, laptop computers, tablet computers, smart phones or other mobile phones, servers, game consoles, televisions, set-top boxes, electronic kiosks, point-of-sale terminals, and/or the like. Each user system 730 may comprise or be communicatively connected to a client application 732 and/or one or more local databases 734.

Platform 710 may comprise web servers which host one or more websites and/or web services. In embodiments in which a website is provided, the website may comprise a graphical user interface, including, for example, one or more screens (e.g., webpages) generated in HyperText Markup Language (HTML) or other language. Platform 710 transmits or serves one or more screens of the graphical user interface in response to requests from user system(s) 730. In some embodiments, these screens may be served in the form of a wizard, in which case two or more screens may be served in a sequential manner, and one or more of the sequential screens may depend on an interaction of the user or user system 730 with one or more preceding screens. The requests to platform 710 and the responses from platform 710, including the screens of the graphical user interface, may both be communicated through network(s) 720, which may include the Internet, using standard communication protocols (e.g., HTTP, HTTPS, etc.). These screens (e.g., webpages) may comprise a combination of content and elements, such as text, images, videos, animations, references (e.g., hyperlinks), frames, inputs (e.g., textboxes, text areas, checkboxes, radio buttons, drop-down menus, buttons, forms, etc.), scripts (e.g., JavaScript), and the like, including elements comprising or derived from data stored in one or more databases (e.g., database(s) 714) that are locally and/or remotely accessible to platform 710. It should be understood that platform 710 may also respond to other requests from user system(s) 730.

Platform 710 may comprise, be communicatively coupled with, or otherwise have access to one or more database(s) 714. For example, platform 710 may comprise one or more database servers which manage one or more databases 714. Server application 712 executing on platform 710 and/or client application 732 executing on user system 730 may submit data (e.g., user data, form data, etc.) to be stored in database(s) 714, and/or request access to data stored in database(s) 714. Any suitable database may be utilized, including without limitation MySQL™, Oracle™, IBM™, Microsoft SQL™, Access™, PostgreSQL™, MongoDB™, and the like, including cloud-based databases and proprietary databases. Data may be sent to platform 710, for instance, using the well-known POST request supported by HTTP, via FTP, and/or the like. This data, as well as other requests, may be handled, for example, by server-side web technology, such as a servlet or other software module (e.g., comprised in server application 712), executed by platform 710.

In embodiments in which a web service is provided, platform 710 may receive requests from user system(s) 730 and/or external system(s) 740, and provide responses in extensible Markup Language (XML), JavaScript Object Notation (JSON), and/or any other suitable or desired format. In such embodiments, platform 710 may provide an application programming interface (API) which defines the manner in which user system(s) 730 and/or external system(s) 740 may interact with the web service. Thus, user system(s) 730 and/or external system(s) 740 (which may themselves be servers), can define their own user interfaces, and rely on the web service to implement or otherwise provide the backend processes, methods, functionality, storage, and/or the like, described herein. For example, in such an embodiment, a client application 732, executing on one or more user system(s) 730, may interact with a server application 712 executing on platform 710 to execute one or more or a portion of one or more of the various functions, processes, methods, and/or software modules described herein.

Client application 732 may be “thin,” in which case processing is primarily carried out server-side by server application 712 on platform 710. A basic example of a thin client application 732 is a browser application, which simply requests, receives, and renders webpages at user system(s) 730, while server application 712 on platform 710 is responsible for generating the webpages and managing database functions. Alternatively, the client application may be “thick,” in which case processing is primarily carried out client-side by user system(s) 730. It should be understood that client application 732 may perform an amount of processing, relative to server application 712 on platform 710, at any point along this spectrum between “thin” and “thick,” depending on the design goals of the particular implementation. In any case, the software described herein, which may wholly reside on either platform 710 (e.g., in which case server application 712 performs all processing) or user system(s) 730 (e.g., in which case client application 732 performs all processing) or be distributed between platform 710 and user system(s) 730 (e.g., in which case server application 712 and client application 732 both perform processing), can comprise one or more executable software modules comprising instructions that implement one or more of the processes, methods, or functions described herein.

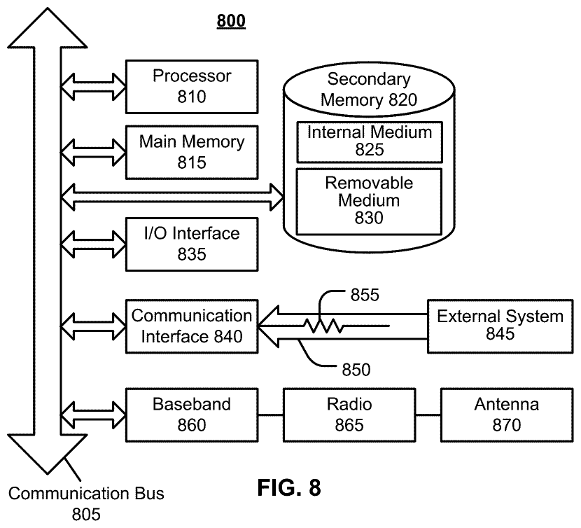

FIG. 8 is a block diagram illustrating an example wired or wireless system 800 that may be used in connection with various embodiments described herein. For example, system 800 may be used as or in conjunction with one or more of the processes, methods, or functions (e.g., to store and/or execute the software) described herein, and may represent components of platform 710, user system(s) 730, external system(s) 740, and/or other processing devices described herein. System 800 can be any processor-enabled device (e.g., server, personal computer, etc.) that is capable of wired or wireless data communication. For example, external control system 102 can be implemented via a processing system 800. Other processing systems and/or architectures may also be used, as will be clear to those skilled in the art.

System 800 may comprise one or more processors 810. Processor(s) 810 may comprise a central processing unit (CPU). Additional processors may be provided, such as a graphics processing unit (GPU), an auxiliary processor to manage input/output, an auxiliary processor to perform floating-point mathematical operations, a special-purpose microprocessor having an architecture suitable for fast execution of signal-processing algorithms (e.g., digital-signal processor), a subordinate processor (e.g., back-end processor), an additional microprocessor or controller for dual or multiple processor systems, and/or a coprocessor. Such auxiliary processors may be discrete processors or may be integrated with a main processor 810. Examples of processors which may be used with system 800 include, without limitation, any of the processors (e.g., Pentium™, Core i7™, Core i9™, Xeon™, etc.) available from Intel Corporation of Santa Clara, California, any of the processors available from Advanced Micro Devices, Incorporated (AMD) of Santa Clara, California, any of the processors (e.g., A series, M series, etc.) available from Apple Inc. of Cupertino, any of the processors (e.g., Exynos™) available from Samsung Electronics Co., Ltd., of Seoul, South Korea, any of the processors available from NXP Semiconductors N.V. of Eindhoven, Netherlands, and/or the like.

Processor(s) 810 may be connected to a communication bus 805. Communication bus 805 may include a data channel for facilitating information transfer between storage and other peripheral components of system 800. Furthermore, communication bus 805 may provide a set of signals used for communication with processor 810, including a data bus, address bus, and/or control bus (not shown). Communication bus 805 may comprise any standard or non-standard bus architecture such as, for example, bus architectures compliant with industry standard architecture (ISA), extended industry standard architecture (EISA), Micro Channel Architecture (MCA), peripheral component interconnect (PCI) local bus, standards promulgated by the Institute of Electrical and Electronics Engineers (IEEE) including IEEE 488 general-purpose interface bus (GPIB), IEEE 696/S-100, and/or the like.

System 800 may comprise main memory 815. Main memory 815 provides storage of instructions and data for programs executing on processor 810, such as any of the software discussed herein. It should be understood that programs stored in the memory and executed by processor 810 may be written and/or compiled according to any suitable language, including without limitation C/C++, Java, JavaScript, Perl, Python, Visual Basic, .NET, and the like. Main memory 815 is typically semiconductor-based memory such as dynamic random access memory (DRAM) and/or static random access memory (SRAM). Other semiconductor-based memory types include, for example, synchronous dynamic random access memory (SDRAM), Rambus dynamic random access memory (RDRAM), ferroelectric random access memory (FRAM), and the like, including read only memory (ROM).

System 800 may comprise secondary memory 820. Secondary memory 820 is a non-transitory computer-readable medium having computer-executable code and/or other data (e.g., any of the software disclosed herein) stored thereon. In this description, the term “computer-readable medium” is used to refer to any non-transitory computer-readable storage media used to provide computer-executable code and/or other data to or within system 800. The computer software stored on secondary memory 820 is read into main memory 815 for execution by processor 810. Secondary memory 820 may include, for example, semiconductor-based memory, such as programmable read-only memory (PROM), erasable programmable read-only memory (EPROM), electrically erasable read-only memory (EEPROM), and flash memory (block-oriented memory similar to EEPROM).

Secondary memory 820 may include an internal medium 825 and/or a removable medium 830. Internal medium 825 and removable medium 830 are read from and/or written to in any well-known manner. Internal medium 825 may comprise one or more hard disk drives, solid state drives, and/or the like. Removable storage medium 830 may be, for example, a magnetic tape drive, a compact disc (CD) drive, a digital versatile disc (DVD) drive, other optical drive, a flash memory drive, and/or the like.

System 800 may comprise an input/output (I/O) interface 835. I/O interface 835 provides an interface between one or more components of system 800 and one or more input and/or output devices. Example input devices include, without limitation, sensors, keyboards, touch screens or other touch-sensitive devices, cameras, biometric sensing devices, computer mice, trackballs, pen-based pointing devices, and/or the like. Examples of output devices include, without limitation, other processing systems, cathode ray tubes (CRTs), plasma displays, light-emitting diode (LED) displays, liquid crystal displays (LCDs), printers, vacuum fluorescent displays (VFDs), surface-conduction electron-emitter displays (SEDs), field emission displays (FEDs), and/or the like. In some cases, an input and output device may be combined, such as in the case of a touch panel display (e.g., in a smartphone, tablet computer, or other mobile device).

System 800 may comprise a communication interface 840. Communication interface 840 allows software to be transferred between system 800 and external devices (e.g. printers), networks, or other information sources. For example, computer-executable code and/or data may be transferred to system 800 from a network server (e.g., platform 710) via communication interface 840. Examples of communication interface 840 include a built-in network adapter, network interface card (NIC), Personal Computer Memory Card International Association (PCMCIA) network card, card bus network adapter, wireless network adapter, Universal Serial Bus (USB) network adapter, modem, a wireless data card, a communications port, an infrared interface, an IEEE 1394 fire-wire, and any other device capable of interfacing system 800 with a network (e.g., network(s) 720) or another computing device. Communication interface 840 preferably implements industry-promulgated protocol standards, such as Ethernet IEEE 802 standards, Fiber Channel, digital subscriber line (DSL), asynchronous digital subscriber line (ADSL), frame relay, asynchronous transfer mode (ATM), integrated digital services network (ISDN), personal communications services (PCS), transmission control protocol/Internet protocol (TCP/IP), serial line Internet protocol/point to point protocol (SLIP/PPP), and so on, but may also implement customized or non-standard interface protocols as well.

Software transferred via communication interface 840 is generally in the form of electrical communication signals 855. These signals 855 may be provided to communication interface 840 via a communication channel 850 between communication interface 840 and an external system 845 (e.g., which may correspond to an external system 740, an external computer-readable medium, and/or the like). In an embodiment, communication channel 850 may be a wired or wireless network (e.g., network(s) 720), or any variety of other communication links. Communication channel 850 carries signals 855 and can be implemented using a variety of wired or wireless communication means including wire or cable, fiber optics, conventional phone line, cellular phone link, wireless data communication link, radiofrequency (“RF”) link, or infrared link, just to name a few.

Computer-executable code is stored in main memory 815 and/or secondary memory 820. Computer-executable code can also be received from an external system 845 via communication interface 840 and stored in main memory 815 and/or secondary memory 820. Such computer-executable code, when executed, enable system 800 to perform the various functions of the disclosed embodiments as described elsewhere herein.

In an embodiment that is implemented using software, the software may be stored on a computer-readable medium and initially loaded into system 800 by way of removable medium 830, I/O interface 835, or communication interface 840. In such an embodiment, the software is loaded into system 800 in the form of electrical communication signals 855. The software, when executed by processor 810, preferably causes processor 810 to perform one or more of the processes and functions described elsewhere herein.

System 800 may comprise wireless communication components that facilitate wireless communication over a voice network and/or a data network (e.g., in the case of user system 730). The wireless communication components comprise an antenna system 870, a radio system 865, and a baseband system 860. In system 800, radiofrequency (RF) signals are transmitted and received over the air by antenna system 870 under the management of radio system 865.

In an embodiment, antenna system 870 may comprise one or more antennae and one or more multiplexors (not shown) that perform a switching function to provide antenna system 870 with transmit and receive signal paths. In the receive path, received RF signals can be coupled from a multiplexor to a low noise amplifier (not shown) that amplifies the received RF signal and sends the amplified signal to radio system 865.

In an alternative embodiment, radio system 865 may comprise one or more radios that are configured to communicate over various frequencies. In an embodiment, radio system 865 may combine a demodulator (not shown) and modulator (not shown) in one integrated circuit (IC). The demodulator and modulator can also be separate components. In the incoming path, the demodulator strips away the RF carrier signal leaving a baseband receive audio signal, which is sent from radio system 865 to baseband system 860.

If the received signal contains audio information, then baseband system 860 decodes the signal and converts it to an analog signal. Then the signal is amplified and sent to a speaker. Baseband system 860 also receives analog audio signals from a microphone. These analog audio signals are converted to digital signals and encoded by baseband system 860. Baseband system 860 also encodes the digital signals for transmission and generates a baseband transmit audio signal that is routed to the modulator portion of radio system 865. The modulator mixes the baseband transmit audio signal with an RF carrier signal, generating an RF transmit signal that is routed to antenna system 870 and may pass through a power amplifier (not shown). The power amplifier amplifies the RF transmit signal and routes it to antenna system 870, where the signal is switched to the antenna port for transmission.

Baseband system 860 is communicatively coupled with processor(s) 810, which have access to memory 815 and 820. Thus, software can be received from baseband processor 860 and stored in main memory 810 or in secondary memory 820, or executed upon receipt. Such software, when executed, can enable system 800 to perform the various functions of the disclosed embodiments.

It will be understood that the benefits and advantages described above may relate to one embodiment or may relate to several embodiments. Aspects described in connection with one embodiment are intended to be able to be used with the other embodiments. Any explanation in connection with one embodiment applies to similar features of the other embodiments, and elements of multiple embodiments can be combined to form other embodiments. The embodiments are not limited to those that solve any or all of the stated problems or those that have any or all of the stated benefits and advantages.

The preceding detailed description is merely exemplary in nature and is not intended to limit the embodiments or the application thereof. The described embodiments are not limited to usage in conjunction with a particular type of radiofrequency ablation systems. Hence, although the present embodiments are, for convenience of explanation, depicted and described as being implemented in a radiofrequency ablation systems, it will be appreciated that it can be implemented in various other types of radiofrequency ablation systems and machines, and in various other systems and environments. Furthermore, there is no intention to be bound by any theory presented in any preceding sections. It is also understood that the illustrations may include exaggerated dimensions and graphical representation to better illustrate the referenced items shown, and are not considered limiting unless expressly stated as such.

What is claimed is:

1. A wireless radiofrequency ablation (RFA) system for medical treatment, comprising:
an implanted system including:
a receiving coil configured to wirelessly receive power, and
one or more electrodes operably coupled to said receiving coil to deliver thermal energy to a target when powered by the wirelessly received power; and
an external control system including:
a radiofrequency (RF) generator, a transmission coil configured to wirelessly transmit power to the receiving coil, and
a user interface configured to allow transmit power parameters to be input in order to control the RF generator.

2. The system of claim 1, wherein the target is a nerve.

3. The system of claim 1, wherein the one or more electrodes are spaced 1-10 mm apart.

4. The system of claim 1, wherein the one or more electrodes are placed in an array and mounted on a flat surface.

5. The system of claim 1, wherein the receiving coil has a diameter of 1-50 mm.

6. The system of claim 1, wherein the one or more electrodes have an annular shape with a diameter of 0.5-10 mm and a length of 1-10 mm.

7. The system of claim 1, wherein the external control system includes an RF generator configured to generate RF signals from 1 Hz to 13.56 MHz.

8. The system of claim 1, wherein the external control system includes an RF switch to multiplex signals to the transmission coil.

9. The system of claim 1, the external control system is configured to measure reflected impedance, calculate a temperature at the target based on the measured reflected impedance, automatically adjust the transmit power parameters based on the calculated temperature.

10. The system of claim 9, wherein the external control system is further configured to receive signals from the implanted system via the transmission coil, and wherein the reflected impedance is measured by analyzing at least one of amplitude, phase and frequency of signals transmitted and received by the transmission coil.

11. The system of claim 1, wherein the one or more electrodes comprise one or more monopolar electrodes configured to deliver energy to the target.

12. The system of claim 11, further comprising a return electrode configured to be positioned on the patient's skin to complete the electrical circuit during ablation.

13. The system of claim 1, further comprising one or more leads operably coupled to the receiving coil and to the electrodes.

14. The system of claim 13, wherein the one or more leads comprise one or more bipolar electrodes configured to deliver energy to the target.

15. The system of claim 13, wherein the one or more leads comprise one or more multipolar electrodes configured to deliver energy to the target.

16. The system of claim 1, wherein the one or more electrodes have an exposed tip diameter of 0.1 mm to 5 mm to provide focused current delivery.

17. The system of claim 1, wherein the external control system is configured to selectively activate individual monopolar electrodes to shape the ablation zone.

18. The system of claim 1, wherein the external control system comprises a microprocessor and memory containing feedback control algorithms.

19. The system of claim 1, wherein the user interface is configured to display feedback parameters including impedance, temperature, system status, and alerts.

20. The system of claim 1, wherein the implanted system further comprises one or more temperature sensors powered by the receiving coil and configured to provide temperature measurements.

21. The system of claim 20, wherein the temperature sensors comprise thermistors incorporated into the electrode leads.

22. The system of claim 1, wherein the external control system includes wireless communication capability for remote monitoring and control.

23. The system of claim 1, integrated with electronic medical records systems and patient management software.

24. The system of claim 1, wherein the implanted system comprises bioresorbable materials.

25. The system of claim 1, wherein the external transmission coil adheres to the skin using an adhesive or tape.

26. The system of claim 1, wherein the implanted receiving coil and leads are coated in a biocompatible polymeric material.

27. The system of claim 1, wherein the inductive coupling between the external transmission coil and receiving coil enhances power transfer efficiency.

28. The system of claim 1, wherein the external control system encodes information in the transmitted power signal.

29. The system of claim 1, wherein the external control system enters a power saving sleep mode when not actively transmitting power.

30. The system of claim 1, wherein the external control system interfaces with interventional imaging systems.

31. The system of claim 30, further comprising utilizing imaging to guide positioning of the electrodes.

32. The system of claim 2, further comprising stimulating the target nerve to confirm placement.

33. A wireless radiofrequency ablation (RFA) system for medical treatment, comprising:
an implanted system including:
a receiving coil configured to wirelessly receive power, and
one or more electrodes operably coupled to said receiving coil to deliver thermal energy to a target when powered by the wirelessly received power; and
an external control system including:
a radiofrequency (RF) generator, a transmission coil configured to wirelessly transmit power to the receiving coil, and
a user interface configured to allow transmit power parameters to be input in order to control the RF generator, wherein the external control system is configured to measure reflected impedance, calculate a temperature at the target based on the measured reflected impedance, automatically adjust the transmit power parameters based on the calculated temperature.

34. The system of claim 33, wherein the external control system is further configured to receive signals from the implanted system via the transmission coil, and wherein the reflected impedance is measured by analyzing at least one of amplitude, phase and frequency of signals transmitted and received by the transmission coil.

35. The system of claim 33, wherein the implanted system further comprises one or more temperature sensors powered by the receiving coil and configured to provide additional temperature measurements.

36. The system of claim 33, wherein the temperature sensors comprise thermistors incorporated into leads of the electrodes.

37. The system of claim 33, wherein the target is a nerve.

38. The system of claim 33, wherein the electrodes are spaced 1-10 mm apart.

39. The system of claim 33, wherein the at least one electrode is placed in an array and mounted on a flat surface.

40. The system of claim 33, wherein the receiving coil has a diameter of 1-50 mm.

41. The system of claim 33, wherein the electrodes have an annular shape with a diameter of 0.5-10 mm and a length of 1-10 mm.

42. The system of claim 33, wherein the external control system includes an RF generator configured to generate RF signals from 1 Hz to 13.56 MHz.

43. The system of claim 33, wherein the external control system includes an RF switch to multiplex signals to the transmission coil.

44. The system of claim 33, wherein the one or more electrodes comprise one or more monopolar electrodes configured to deliver energy to the target.

45. The system of claim 43, further comprising a return electrode configured to be positioned on the patient's skin to complete the electrical circuit during ablation.

46. The system of claim 33, wherein the one or more electrodes have an exposed tip diameter of 0.1 mm to 5 mm to provide focused current delivery.

47. The system of claim 33, wherein the one or more electrodes are monopolar electrodes and wherein the external control system is configured to selectively activate individual monopolar electrodes to shape the ablation zone.

48. The system of claim 33, wherein the external control system comprises a microprocessor and memory containing feedback control algorithms.

49. The system of claim 33, wherein the user interface is configured to display feedback parameters including impedance, temperature, system status, and alerts.

50. The system of claim 33, wherein the external control system includes wireless communication capability for remote monitoring and control.

51. The system of claim 33, integrated with electronic medical records systems and patient management software.

52. The system of claim 33, wherein the implanted system comprises bioresorbable materials.

53. The system of claim 33, wherein the external transmission coil adheres to the skin using an adhesive or tape.

54. The system of claim 33, wherein the implanted receiving coil and leads are coated in a biocompatible polymeric material.

55. The system of claim 33, wherein the inductive coupling between the external transmission coil and receiving coil enhances power transfer efficiency.

56. The system of claim 33, wherein the external control system encodes information in the transmitted power signal.

57. The system of claim 33, wherein the external control system enters a power saving sleep mode when not actively transmitting power.

58. The system of claim 33, wherein the external control system interfaces with interventional imaging systems.

59. The system of claim 58, further comprising utilizing imaging to guide positioning of the electrodes.

60. A method for radiofrequency ablation comprising:
positioning and aligning transmission coil over implanted receiving coil;
perform sensory nerve and motor nerve testing to verify an electrode placement is not adjacent to tissue or nerves not requiring ablation;
setting parameters of an external control system, including a time period and a target temperature;
initializing the external control system, wherein initializing the system comprises setting an initial power increase amount and an initial time interval;
measuring reflected impedance; and
if reflected impedance is greater than a first threshold, increase power by set amount every interval,
if the reflected impedance is between a second threshold and the first threshold, then reducing the power increase amount by a set amount and reducing the time interval, and
if reflected impedance is below the second threshold, holding the power constant;
maintaining a constant power for the time period once reflected impedance is equal to or below the second threshold; and
reducing power and monitoring impedance rise to ensure cooling before removing transmission coil.

61. The method of claim 60, wherein the receiving coil is located 1-15 cm from a target nerve.

62. The method of claim 60, wherein parameters comprise a 90 second time period and an 80° C. target temperature.

63. The method of claim 60, wherein initializing the system comprises measuring an initial reflected impedance and setting an initial power.

64. The method of claim 60, wherein the initial power is about 1 W.

65. The method of claim 60, wherein the first threshold is about 11Ω.

66. The method of claim 60, wherein the second threshold is about 9Ω.

67. The method of claim 60, wherein the initial power increase amount is about 0.5 W.

68. The method of claim 60, wherein the initial time interval is about 5 sec.
